(19) United States
(12) Patent Application Publication
(10) Pub. No.: US 2004/0177848 A1
(43) Pub. Date: Sep. 16, 2004

(54) Multi-compartment inhaler

(76) Inventor: Kenneth A. Alley, Berwick, PA (US)

Correspondence Address: LAW OFFICES OF MARK A. GARZIA, P.C., 2058 CHICHESTER AVE, BOOTHWYN, PA, 19061, US

(21) Appl. No.: 10/799,059

(22) Filed: Mar. 12, 2004

Related U.S. Application Data
(60) Provisional application No. 60/453,855, filed on Mar. 12, 2003.

(51) Int. Cl. A61M 11/00; A61M 15/00; A61M 16/00

(52) U.S. Cl. 128/200.14; 128/200.23; 128/203.15

(57) ABSTRACT

An inhaler (drug delivery system) that incorporates more than one drug storage chamber and provides the means to dispense a controlled dose of more than a single drug during or/in a single inhalation activity/event. The present invention also provides an improved drug dosing control mechanism and air flow evacuation system that has the means to isolate each individual drug during delivery and is user-friendly.

### CROSS REFERENCE TO RELATED APPLICATIONS

The present application claims the benefits under 35 U.S.C. §119(e) of U.S. Provisional Application No. 60/453,855 filed Mar. 12, 2003, titled Single, Double or Multiple Drug Dosing and Air Flow assembly for Dry Powder Inhalers in the name of Kenneth A. Alley.

U.S. Provisional Application No. 60/453,855 filed Mar. 12, 2003, is hereby incorporated by reference as if fully set forth herein.

### FIELD OF THE INVENTION

The present invention relates generally to inhalation devices that are commonly used by patients who require inhalers as a drug delivery method. More specifically, the present invention is an inhaler that incorporates more than one drug storage chamber, and simultaneously provides the means to dispense a controlled dose of more than a single drug during or/in a single inhalation activity/event. The present invention also provides an improved drug dosing control mechanism that is user-friendly.

### BACKGROUND OF THE INVENTION

There are a host of inhalers on the market that are used for the purpose of delivering lifesaving drugs. One of the most common uses for inhalers, are for delivering medications that are targeted and delivered into the air passageways and lungs of those who suffer from asthma. In order for these prescribed medications to work as a treatment for disease they must be capable of being delivered through an inhalation device. In order to deliver the medications to the lungs and air passage-ways the medications must be substantially small in size, mass and compatible in composition to efficiently flow through the inhalation device and into the patient's lungs.

Many of the existing medications which are delivered through the use of an inhaler require substantial expense and technical capability to manufacture. Some of these medications are complex compounds that not only require expensive processing but in many cases, larger doses are required to compensate, due to the drug delivery failure rate per inhalation event. Some complex medications are compounds and are larger in molecular mass and size, thus a percentage of the dose will be absorbed within the user's mouth and not delivered to the targeted sight.

One of the most successful inhalers on the market is manufactured by Astra Pharma Inc., brand name Turbuhaler. This particular inhaler incorporates four small dosing wells that are filled with a single medication. During inhalation the air must flow from underneath the drug-well up through the mouthpiece. If the Turbuhaler were to be jarred or dropped prior to inhalation, a portion of the drug could easily fall downward into the dead space of the device thus, preventing the delivery of the prescribed dose.

Additionally, there are limitations to developing future drugs for inhalation use, as a result of active chemicals being incompatible with one another. In many cases the stability of a compound may be negatively affected and therefore the shelf life of such medications would be too short or incompatible with one another to have any practical use. Additionally, the technology to combine or manufacture complex compounds/clusters may not exist or would be too expensive and unreliable to produce. In many cases, drug clusters must be created to manufacture medications thus, creating other obstacles (including larger molecular masses of the drug candidates) that negatively affect the function and efficacy of the inhaler.

Accordingly, there is a need for an inhaler that incorporates more than one drug storage chamber, and that simultaneously provides the means to dispense a controlled dose of more than a single drug during or/in a single inhalation activity/event.

### SUMMARY OF THE INVENTION

The present invention is an inhaler device that has more than one drug storage chamber, and simultaneously provides the means to dispense a controlled dose of more than a single drug during a single inhalation activity/event (i.e., when the user inhales and the medicine exits the inhaler and enters the user's mouth or nose, etc.). The present invention also provides an improved drug dosing control mechanism that has the means to isolate each individual drug during delivery and is user-friendly.

As previously explained, there would be substantial advantages to having the ability to isolate specific drugs, chemicals or compounds within separate drug chambers within a single inhaler device. For example, a few advantages include the cost of drug production (less expensive processing), smaller molecular compounds or drugs could be used, thus improving, the efficiency of the drug delivery device and quantity of the drug delivered to the target (air passageways and lungs). Additionally, future drug development, drug testing and compatibility issues would be enhanced by such a device, thus enhancing shelf life, product stability, etc. Drug cocktails could be developed that otherwise would not be practical to incorporate into a single use inhaler device.

Another unique feature of this invention is that it provides an improved drug dosing control mechanism. Unlike the other inhalers previously available, the present invention incorporates an isolated single (or more than one) dosing disc that is manipulated within, or communicates with separate drug chambers and if desired a mouthpiece that isolates each individual drug passageway during each inhalation activity/event.

In the present invention, the dosing or drug wells are evacuated by multiple air passageways, thus providing a very efficient and complete evacuation of the drug well. Additionally, if the present invention were dropped or jarred, the medication will fall directly into an air flow channel that will be evacuated during the inhalation activity/event, thus delivering the prescribed amount of medication. The increased air volume and biaxial air flow in and around the dosing-well, will improve the evacuation of the drug or drugs from the dosing-well and the overall inhalation device.

It is important to emphasize that there are numerous mechanical configurations to dispense a given drug from an inhaler. This particular design may be adapted to numerous configurations of inhalers. It is not the goal of this invention to limit the scope to any one particular dosing control mechanism, and/or air flow system.

It is the goal of this invention to emphasize, reiterate and claim, this invention teaches and claims a novel new ihaler that adapts two or more drug storage chambers (permanent or refillable or dry powder or aerosol/mist/liquid) within the inhaler, that will/may simultaneously provide means to dispense a controlled dose of two or more drugs during or/in a single inhalation activity/event.

This novel inhaler technology will provide new opportunities within the scope of existing inhaled drugs (improved stabilities, manufacturing cost, device function, etc.) and equally, if not more important, it will open the door to new drug cocktails, clusters and new drug research and developments thus, by removing compatibility, stability and manufacturing limitations that exist with current inhaler technology. Previously incompatible drugs, agents, compounds or clusters could be isolated from one another to create a single inhaler with customized drug delivery and multi-disease treatment within a single inhaler device.

### BRIEF DESCRIPTION OF THE DRAWINGS

The foregoing summary, as well as the following detailed description, may be better understood when read in conjunction with the accompanying drawings, which are incorporated in and form a part of the specification. The drawings serve to explain the principles of the invention and illustrate embodiments of the present invention that are preferred at the time the application was filed. It should be understood however that the invention is not limited to the precise arrangements and instrumentalities shown.

FIG. 1 is an exploded side view of an inhaler device's dosing control mechanism in accordance with the present invention.

FIG. 2 is an isometric view of the multiple drug storage chamber platform adapted to an inhaler in accordance with the present invention.

FIG. 3 is an isometric view of a dosing well disc with multiple and metered drug dosing wells adapted in accordance with the present invention.

FIG. 4 is an isometric view of a multi-chambered air passageway platform in accordance with the present invention.

FIG. 5 is an isometric view of an alternative embodiment of the inhaler device's dosing control mechanism in accordance with the present invention.

FIG. 6 is a side view of the dosing well disc communicating with the multi-chambered air passageway platform.

### DETAILED DESCRIPTION OF THE PREFERRED EMBODIMENT

As previously mentioned there are numerous inhaler designs that could be employed to include the multi-drug chambers and multi-drug dispenser technology within a single inhaler device disclosed in the present specification. Additionally, the mechanical system required to physically control the motion of the inhaler parts could also be, of numerous designs and therefore are not the main scope or claim of this invention.

Referring now to FIG. 1, an inhaler dosing control mechanism in accordance with the present invention is indicated at 10. In a preferred embodiment, the inhaler device is designed to incorporate and/or isolate more than one drug storage chamber and simultaneously provide the means to dispense a controlled dose of more than a single drug during or/in a single inhalation activity/event.

In a preferred embodiment, the apparatus 10 comprises four primary parts; namely, an inhaler mouthpiece 11, a multiple drug storage chamber platform 20, a dosing-well disc 30 with multiple and metered drug dosing-wells, and a multi-chambered air passageway platform 40, as illustrated in FIGS. 1-4.

In the illustrated embodiment, the disc 30 may be manipulated, preferably in a rotating manner, with respect to the multiple drug storage chambers platform 20 and the multi-chambered air passageway platform 40. The disc 30 may be designed with a diameter slightly larger than its neighboring parts to allow the user to manipulate the disc more easily. Other ways, not shown in the drawings, may be designed to manipulate the dosing-well disc 30 with respect to both the storage chamber platform 20 and the multi-chambered air passageway platform 40. A simple thumb-wheel with a plurality of small projections or other well-known means for moving the disc 30 may be incorporated. For example, U.S. Pat. No. 5,582,162 to Peterson discloses a pawl/cam follower advancement mechanism that is activated by a trigger.

In the embodiment illustrated, the inhaler 10 allows the different drugs/medicaments in the chambers to mix in the mouthpiece 11 during the evacuation process. However, the inhaler mouthpiece 11 may also be designed in such a way to isolate each individual drug passageway as the user of the device evacuates the prescribed dose. A divider wall (not shown) could be incorporated that would provide a separate exit passageway for each different drug chamber. Additionally, mouthpiece 11 may be designed to snap off for cleaning purposes and/or for providing access to the drug storage chambers. It may be desired to incorporate refillable cartridges or disposable drug storage disc that could be adapted to incorporate the features of this invention.

Referring now to FIG. 2, multiple drug storage chamber platform 20 includes drug storage chambers 22, air flow bypass grooves 24, and exit port 26. The drugs/medicaments are stored in the drug storage chambers during the manufacturing process. The drugs remain sealed in the chambers 22 until the user is ready to use the inhaler 10. The drugs/medicaments stored in the chambers 22 can be a powder, liquid, vapor or any state that typical inhalers are used to deliver drugs.

There must be at least one drug storage chamber 22. A person skilled in the art, after reading the present disclosure, would understand that two or more chambers 22 may easily be incorporated into the drug storage platform 20.

One aspect that distinguishes the present invention from previous inhalers is that two or more drugs may be delivered in a single evacuation event. The illustrated embodiment shows three drug storage chambers 22; this will allow for up to three different drugs/medicaments to be delivered to the user. Of course depending on the medicaments, two of the chambers 22 may be filled with one drug and the remaining chamber may be filled with a different drug, if that is the ratio prescribed to the user.

In the preferred embodiment, the drug storage chambers 22 would be filled with the prescribed drugs during the manufacturing process. It should be noted that in another embodiment, drug storage chambers 22 could be replaced by a disposable and/or refillable drug cartridges. Further, it is known in the art to place the stored drugs under pressure during the manufacturing process, if required.

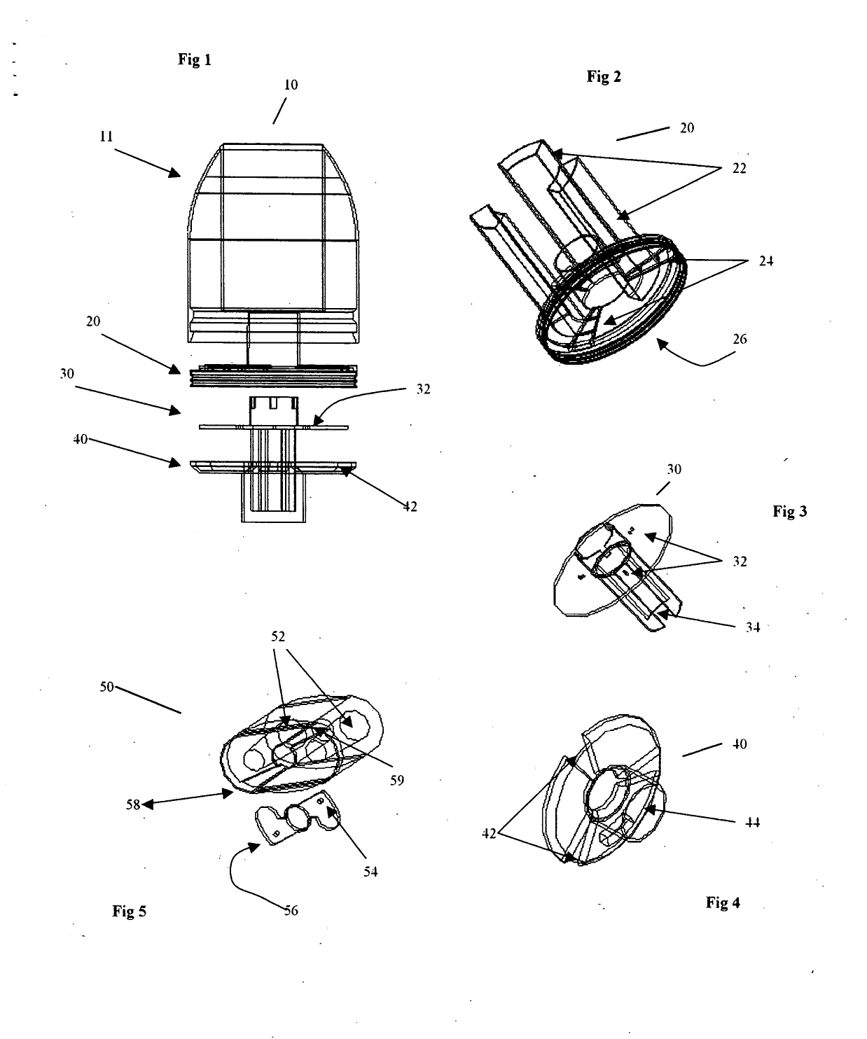

Referring now to FIG. 3, dosing-well disc 30 has multiple and metered drug dosing-wells 32, and positioning and gear mechanism 34. In the final assembly, the dosing well disc 30 is sandwiched between the multiple drug storage platform 20 and the multi-chambered air passageway platform 40. The positioning and gear mechanism 34 will extend downward through opening 44 of the multi-chambered air passageway platform 40 (i.e., generally away from the mouthpiece 11) thus providing a means for rotating the dosing well disc 30 with respect to the multiple drug storage chambers platform 20 and the multi-chambered air passageway platform 40. The control gear mechanism is preferably rotated (either continuously clockwise, continuously counter-clockwise, or first one direction then the opposite direction) to operate the inhaler.

The dosing wells 32 are designed to perform two functions. First, they are precisely sized to withdraw the prescribed amounts of medicaments from the chambers 22 when the disc is manipulated; second, they temporarily hold the prescribed amount of medicaments until the evacuation event occurs.

Referring now to FIG. 4, the air passageway platform includes a plurality of air passageway grooves 42 and an opening 44. The number of air passageway grooves 42 usually equal the number of drug storage chambers 22 in the drug storage platform 20. However, depending on the type of medicaments and the prescribed amounts of medicaments needed to be delivered to the user, there may be more grooves 42 than chambers 22.

As previously mentioned, the mechanical system required to physically control the motion of the inhaler parts, and/or more specifically, the movement of the dosing-well disc 30 with respect to the multiple drug storage platform 20 and multi-chambered air passageway platform 40 could adapt or include numerous designs and therefore are not the main scope of this invention.

The operation of the subject inhaler 10 will be described with reference to FIG. 6 in which is illustrated the disc 30 being sandwiched between the storage platform 20 and the passageway platform 40. During the manufacturing process, the chambers 22 are filled with medicaments and sealed therein. The storage platform 20 (with the medicaments stored in chambers 22) is inserted into nosepiece 11. Disc 30 is then placed next to storage platform 20, and air-passageway platform 40 is snapped onto storage platform 20, allowing disc 30 to move in a controlled manner with respect to both the storage platform 20 and the air- passageway platform 40. Alternative assembly methods may be used depending on the types of medicaments used, reduce costs, or in order to streamline the assembly process.

When the inhaler is manufactured, the disc 30 abuts the end of the chambers 22, thereby sealing one end of the chambers 22 and preventing the medicaments from escaping. In the initial step, the disc 30 is rotated so that the dosing wells 32 are aligned directly under the chambers 22. The dosing wells 32 are made so that their diameter and depth allows a pre-determined amount (i.e., the prescribed amount) of medicaments to leave each chamber 22, to be temporarily stored in the dosing wells 32.

In the inhalation position, the disc 30 is manipulated further so that the metered dosing wells 32 will be aligned directly above the air passageway channels 42 and directly below the air flow bypass grooves 24 of the multiple drug storage chambers platform 20. During the inhalation event the metered drug dosing-wells 32 are evacuated by the movement of air through multiple air passageways 68, 64, 62 and 66, thus providing a very efficient and complete evacuation of the metered drug dosing-well 32.

Another aspect of the present invention is that the dosing wells 32 are evacuated by multiple air passageways, thus providing a very efficient and complete evacuation of the metered drug dosing-well. Biaxial air flow (as illustrated in two-dimensional FIG. 6, biaxial refers to the X and Y planes) in and around the dosing-wells, will improve the evacuation of the drug or drugs from the dosing-wells 32 and the improve the overall efficiency of the instant inhaler 10.

Additionally, if the present invention were dropped or a jarred the medication will fall directly into an air flow channel(s) 42 that will be evacuated during the inhalation activity/event by air flow 64, thus delivering the prescribed amount of medication. The increased air volume and biaxial air flow in and around the dosing-wells, will improve the evacuation of the drug or drugs from the dosing-well and the overall inhaler 10.

During reloading, the disc 30 is rotated and the metered dosing wells 32 are repositioned again directly below the drug storage chambers 22. When the dosing wells 32 are aligned with the storage chambers 22, they are simultaneously misaligned with the multi-chambered air passageway channels 42. In this particular position, the prescribed drug/drugs will fill their respective metered dosing-wells 32, and stay there until the disc 30 is manipulated again thereby closing off the chambers 22 and allowing the drugs to communicate with the passageway channels 42. This process will repeat itself every time the inhaler 10 is used.

In some cases, it may be desirable to completely isolate the medicaments as they are delivered to the user. The air passageway platform 40 may be designed to restrict the movement of disc 30 so that only the medicament from one chamber 22 will drop into its respective dosing well 32 and respective passageway channel 42. The inhaler mouthpiece 11 may also be designed in such a way to isolate each individual drug passageway as the user of the device evacuates the prescribed dose. A divider wall (not shown) could be incorporated that would provide a separate exit passageway for each different drug chamber. These modifications will allow an inhaler embodiment that has individual drug passageways during each inhalation activity/event thereby preventing the commingling of medicaments during delivery.

Mouthpiece 11 may be designed to snap off for cleaning purposes and/or for providing access to the drug storage chambers. It may be desired to incorporate refillable cartridges or disposable drug storage disc that could be adapted to incorporate the features (multiple and isolated drugs) of this invention.

An aspect of this invention is, that unlike previous inhalers, the use of two or more drug storage chambers (permanent or refillable or dry powder or aerosol/mist/liquid) within the inhaler, that can simultaneously dispense a controlled dose of two or more drugs during a single inhalation activity/event.

It would be apparent to one skilled in the art, after reading the present disclosure, to include a plurality of discs 30 to manipulate the dosing of each medicament. The discs would communicate with each other during pre-determined manipulations to alternately align with the chambers 22, and then align with the passageway channels 42. For example, during the loading process, discs 30 may be first turned ¼ turn clockwise, allowing one set of dosing wells 32 to fill with medicaments; then discs 30 may be allowed to rotate ½ turn counter-clockwise to allow the medicaments to drop into a first set of air passageway channels 42, while a second set of dosing wells 32 fill with other medicaments. A final rotation (i.e., ¼ turn counterclockwise or ¾ turn clockwise) of discs 30 will allow the medicaments in the second set of dosing wells 32 to drop down into a second set of passageway channels 42. Then upon the evacuation event, the medicaments in both the first and second set of passageway channels 42 will be delivered to the user. The inhaler 10 would be designed to allow the discs 30 only one combination of turns to prevent overdosing or underdosing; this can be done with a set of nibs and detents that click/grab during the pre-determined rotations.

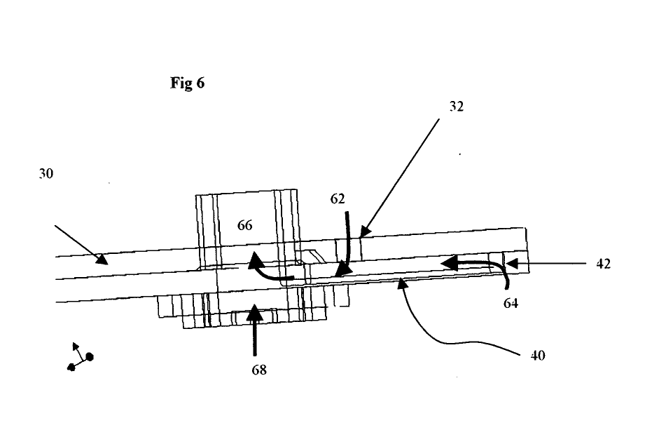

FIG. 5 is alternative embodiment of the subject inhaler 50 that incorporates two different drug storage chambers 52. This particular design includes metered drug wells 54 of drug dosing disc 56. This particular design combines the previously described multi-chambered air passageway platform 40 and the dosing disc 30, into a single piece thus, minimizing the required components of the inhaler assembly (and reducing the assembly costs). It also includes the drug chambers within the housing of the device. These particular chambers could be adapted to accept a cartridge and or aerosol combination.

A unique feature of this embodiment, would be the combination of a dry powder drug with a pressurized chamber to help evacuate the drug and/or to include an additional aerosol medication simultaneously with the dry powder.

For example, a pressurized air would be referred to as one of the drugs, thus in the multi-drug inhaler technology. The air could be incorporated to flow behind and or through the metered dosing well, thus aiding in the function evacuation and delivery of the prescribed medications.

A unique feature of an alternative embodiment, would be the combination of a dry powder drug with a pressurized chamber to help evacuate the drug and/or to include an additional aerosol medication simultaneously with the dry powder. A pressurized air supply would be incorporated into the inhaler to help evacuate the drugs. The air could be incorporated to flow behind and/or through the metered dosing-wells, thus aiding in the function evacuation and delivery of the prescribed medications.

Although this invention has been described and illustrated by reference to specific embodiments, it will be apparent to those skilled in the art that various changes and modifications may be made which clearly fall within the scope of this invention. The present invention is intended to be protected broadly within the spirit and scope of the appended claims.

I claim:

1. An inhaler for delivering drugs/medications by inhalation, the inhaler device comprising of:
a) a housing defining an inlet, an outlet spaced from said inlet, and an airflow path from said inlet to said outlet;
b) a plurality of storage chambers for storing a plurality of medications;
c) means that communicate with said drug storage chambers for withdrawing a pre-determined amount of medication from one or more chambers; and
d) means to simultaneously dispense said withdrawn medication during a single inhalation activity/event.

2. The inhaler of claim 1 wherein said outlet comprises a mouthpiece.

3. The inhaler of claim 2 wherein said mouthpiece isolates each individual drug passageway during each inhalation activity/event.

4. The inhaler of claim 2 wherein said mouthpiece includes a plurality of drug passageways for isolating each individual medication as the user of the device evacuates the prescribed dose.

5. The inhaler of claim 1 wherein said inhaler comprises a plurality of dosing discs.

6. The inhaler of claim 1 where said inhaler further comprises a plurality of channels for isolate said medications until they exit the outlet during the evacuation process.

7. The inhaler of claim 1 where said inhaler includes a combination of a dry powder drug with a pressurized chamber to help evacuate the drug and/or to include an additional aerosol medication simultaneously with the dry powder.

8. An inhaler for delivering drugs/medications by inhalation, the inhaler device comprising of:
a) a housing defining an inlet, an outlet spaced from said inlet, and an airflow path from said inlet to said outlet;
b) a plurality of storage chambers for storing a plurality of medications;
c) a rotatable dosing disc having a plurality of dosing wells that communicate with said drug storage chambers for withdrawing a pre-determined amount of medication from one or more chambers; and
d) air passageway platform having a plurality of passageway channels, said air passageway channels communicating with both the dosing wells and said outlet of the mouthpiece to define multiple air passageways for delivering said withdrawn medication during a single inhalation activity/event.

9. The inhaler of claim 8 wherein said multiple air passageways allow biaxial air flow to move the medicaments through the inhaler and out the outlet.

10. The inhaler of claim 9 further comprising pressurized cartridges that communicate with the dosing wells for ensuring the complete evacuation of the medicaments from the wells, the air passageways and the outlet during the inhalation event.
